(19) United States
(12) Patent Application Publication
(10) Pub. No.: US 2012/0330235 A1
(43) Pub. Date: Dec. 27, 2012

(54) Drug Delivery Device Including a Dynamic Drug Reservoir and Ergonomic User Interface

(75) Inventors: Ben J. Moga, Houston, TX (US); Jeremy Ling, Saint Paul, MN (US); Jason Gerold, Shakopee, MN (US)

(73) Assignee: RATIO, INC., Monona, WI (US)

(21) Appl. No.: 13/532,191

(22) Filed: Jun. 25, 2012

Related U.S. Application Data
(60) Provisional application No. 61/500,364, filed on Jun. 23, 2011.

(51) Int. Cl. A61M 5/20

(52) U.S. Cl. 604/131

(57) ABSTRACT

A drug delivery device includes a housing with a base and a cover that define an enclosure. A drug container disposed within the enclosure includes a drug reservoir configured to retain the drug therein. The drug container includes external features formed on an exterior of the drug container configured to interface with the housing so as to allow for dynamic movement relative to the housing to relieve stress on the rigid or flexible drug container when the needle assembly is actuated between the engaged and unengaged configurations. The drug delivery device includes a needle assembly with a needle and a drug channel connected between the drug container and needle assembly. A needle actuation mechanism is coupled to the needle assembly and actuatable between an engaged configuration and an unengaged configuration. The needle is extended from the housing in the engaged configuration and retracted in the housing in the unengaged configuration.

### TECHNICAL FIELD

Various embodiments disclosed herein relate to drug delivery devices. More particularly, certain implementations relate to a drug delivery device including a dynamic drug reservoir and an ergonomic user interface.

### BACKGROUND

In certain circumstances, it is desirable to inject medication directly into human tissue. Syringes are used to inject pharmaceuticals into tissue areas, such as the intramuscular tissue layer, the subcutaneous tissue layer, and the intradermal tissue layer. Each of these tissue layers has specific characteristics that affect the amount of fluid pressure needed to inject a fluid into the targeted layer. When injecting fluids into each of these layers, the user exerts enough force on the injection device to overcome different amounts of back pressure associated with the particular tissue layer.

Manual syringes and auto-injection devices are devices that are designed to administer a fluid-flowing drug subcutaneously. For example, injection pens may include a pharmaceutical reservoir and a disposable hollow-core needle through which the pharmaceutical is injected into the tissue. The injection is performed with an injection pen by contacting the injection pen to the skin at the injection site and pressing a button that first inserts the needle using a spring into the subcutaneous tissue and then injects the pharmaceutical to the subcutaneous tissue. However, pen injectors typically require the user to input kinetic energy to maintain the needle in a delivery state in the tissue.

### SUMMARY

Disclosed herein are embodiments of a drug delivery device including a drug container that moves within the enclosure when the needle is engaged, thereby minimizing the size of the drug delivery device and relieving stress on components of the device. In some embodiments, the device is further configured to activate a pressure source to deliver the drug when the needle is engaged.

In Example 1, a drug delivery device for delivering a drug to an individual includes a housing with a base and a cover that define an enclosure. A drug container is disposed within the enclosure. The base includes a track assembly or other means for allowing dynamic movement of the drug container relative to the housing or enclosure. The drug delivery device further includes a needle assembly including a needle and a rigid or flexible drug channel fluidly connected between the drug container and needle assembly. The drug container includes a drug reservoir configured to retain the drug therein and may contain guide features formed on an exterior of the drug container configured to interface with the housing so as to relieve stress on the drug channel when the needle actuation mechanism is actuated between the engaged and unengaged configurations. A needle actuation mechanism is coupled to the needle assembly and is actuatable between an engaged configuration and an unengaged configuration. The needle is extended from the housing in the engaged configuration and retracted in the housing in the unengaged configuration.

In Example 2, the drug delivery device according to Example 1, wherein a major axis of the drug channel is substantially parallel to a major surface of the drug container when the needle actuation mechanism is in the engaged configuration, and wherein the major axis of the drug channel is non-zero angled with respect to the major surface of the drug container when the needle actuation mechanism is in the unengaged configuration.

In Example 3, the drug delivery device according to either Example 1 or 2, and further comprising a pressure source engagable with the drug container for urging the drug from the reservoir through the needle assembly via the drug channel.

In Example 4, the drug delivery device according to any of Examples 1-3, and further comprising a pressure activation assembly coupled to the needle actuation mechanism, wherein actuation of the needle actuation mechanism to the engaged configuration causes a presenting end of the pressure activation assembly to activate the pressure source.

In Example 5, the drug delivery device according to any of Examples 1-4, wherein the pressure source comprises a hydrogel that expands in response to exposure to a fluid.

In Example 6, the drug delivery device according to any of Examples 1-5, wherein actuation of the needle actuation mechanism to the engaged configuration causes the presenting end to release fluid that activates the hydrogel.

In Example 7, the drug delivery device according to any of Examples 1-6, wherein the needle actuation mechanism rotates about an axis when actuated between the unengaged and engaged configurations.

In Example 8, the drug delivery device according to any of Examples 1-7, wherein the needle actuation mechanism comprises an adapter that couples to the needle assembly and is configured to convert rotational motion of the needle actuation mechanism to translational motion of the needle assembly.

In Example 9, the drug delivery device according to any of Examples 1-8, wherein the needle actuation mechanism comprises an ergonomic lever that is lockable in the engaged and unengaged configurations and freely slidable between the engaged and unengaged configurations.

In Example 10, the drug delivery device according to any of Examples 1-9, wherein the drug channel comprises a flexible polymeric material.

In Example 11, the drug delivery device according to any of Examples 1-10, wherein the needle is the only metallic component of the drug delivery device.

In Example 12, the drug delivery device according to any of Examples 1-11, wherein the ergonomic lever is self-locking.

In Example 13, a drug delivery device for delivering a drug to an individual includes a housing defining an enclosure having a top surface, a bottom surface, and one or more side walls extending between the top and bottom surfaces. A drug container disposed within the enclosure includes a drug reservoir for retaining the drug therein. The drug container is configured to cooperate with the housing to allow for dynamic movement of the drug container relative to the housing. The drug delivery device further includes a needle assembly including a needle and a drug channel fluidly connected between the drug container and needle assembly. A needle actuation mechanism is coupled to the needle assembly and is actuatable between an engaged configuration and an unengaged configuration. The needle is extended from the housing in the engaged configuration and the needle is retracted in the housing in the unengaged configuration. The drug container is substantially unconstrained within the drug delivery device so as to relieve stress on the drug channel when the needle actuation mechanism is actuated between the engaged and unengaged configurations.

In Example 14, the drug delivery device according to Example 13, wherein a major axis of the drug channel is substantially parallel to a major surface of the drug container when the needle actuation mechanism is in the engaged configuration, and wherein the major axis of the drug channel is non-zero angled with respect to the major surface of the drug container when the needle actuation mechanism is in the unengaged configuration.

In Example 15, the drug delivery device according to either Example 13 or 14, and further comprising a pressure source engagable with the drug container for urging the drug from the reservoir through the needle assembly via the drug channel.

In Example 16, the drug delivery device according to any of Examples 13-15, and further comprising a pressure activation assembly coupled to the needle actuation mechanism, wherein actuation of the needle actuation mechanism to the engaged configuration causes a presenting end of the pressure activation assembly to activate the pressure source.

In Example 17, the drug delivery device according to any of Examples 13-16, wherein the pressure source comprises a hydrogel that expands in response to exposure to a fluid.

In Example 18, the drug delivery device according to any of Examples 13-17, wherein actuation of the needle actuation mechanism to the engaged configuration causes the presenting end to release fluid that activates the hydrogel.

In Example 19, the drug delivery device according to any of Examples 13-18, wherein the needle actuation mechanism rotates about an axis when actuated between the unengaged and engaged configurations.

In Example 20, the drug delivery device according to any of Examples 13-19, wherein the needle actuation mechanism comprises an adapter that couples to the needle assembly and is configured to convert rotational motion of the needle actuation mechanism to translational motion of the needle assembly.

In Example 21, the drug delivery device according to any of Examples 13-20, wherein the needle actuation mechanism comprises an ergonomic lever that is lockable in the engaged and unengaged configurations and freely slidable between the engaged unengaged configurations.

In Example 22, the drug delivery device according to any of Examples 13-21, wherein the drug channel comprises a flexible polymeric material.

In Example 23, the drug delivery device according to any of Examples 13-22, wherein the needle is the only metallic component of the drug delivery device.

In Example 24, a drug delivery device for delivering a drug to an individual includes a housing defining an enclosure and a drug container disposed within the enclosure comprising a drug reservoir for retaining the drug therein. The drug delivery device further includes a needle assembly including a needle and a drug channel fluidly connected between the drug container and needle assembly. A pressure source is engagable with the drug container for urging the drug from the reservoir through the needle assembly via the drug channel. A needle actuation mechanism is coupled to the needle assembly and is manually actuatable between an engaged configuration and an unengaged configuration. The needle is extended from the housing in the engaged configuration and retracted in the housing in the unengaged configuration. The needle actuation mechanism is manually lockable in the engaged and unengaged configurations and manually slidable between the engaged and unengaged configurations. A pressure activation assembly is coupled to the needle actuation mechanism such that manually actuation of the needle actuation mechanism to the engaged configuration causes a presenting end of the pressure activation assembly to activate the pressure source.

In Example 25, the drug delivery device according to Example 24, wherein the pressure source comprises a hydrogel that expands in response to exposure to a fluid.

In Example 26, the drug delivery device according to either Example 24 or 25, wherein actuation of the needle actuation mechanism to the engaged configuration causes the presenting end to release fluid that activates the hydrogel.

In Example 27, the drug delivery device according to any of Examples 24-26, wherein the drug container configured to cooperate with the housing to allow for guided movement of the drug container substantially parallel to a bottom surface of the housing.

In Example 28, the drug delivery device according to any of Examples 24-27, wherein the drug container slides along the bottom surface to relieve stress on the drug channel when the needle actuation mechanism is actuated between the engaged and unengaged configurations.

While multiple embodiments are disclosed, still other embodiments of the present invention will become apparent to those skilled in the art from the following detailed description, which shows and describes illustrative embodiments of the invention. Accordingly, the drawings and detailed description are to be regarded as illustrative in nature and not restrictive.

### BRIEF DESCRIPTION OF THE DRAWINGS

FIG. 1 is a perspective view of an embodiment of a drug delivery device in an unengaged configuration.

FIG. 2 is a cross-sectional view of the drug delivery device shown in FIG. 1.

FIG. 3 is a perspective cross-sectional view of the drug delivery device shown in FIG. 1.

FIG. 4 is a cross-sectional view of the drug delivery device shown in FIG. 1 through a pressure activation assembly.

FIG. 5 is a perspective view of an embodiment of the drug delivery device in an engaged configuration.

FIG. 6 is a cross-sectional view of the drug delivery device shown in FIG. 5.

FIG. 7 is a perspective cross-sectional view of the drug delivery device shown in FIG. 5.

FIG. 8 is a perspective view of an embodiment of a base portion of the housing of the drug delivery device.

FIG. 9A is front view and FIG. 9B is a side view of a needle assembly adapter for coupling the needle assembly to the needle actuation mechanism in the drug delivery device.

FIG. 10 is a cross-sectional view illustrating the coupling relationship between the needle assembly adapter and needle actuation mechanism.

FIG. 11 is a perspective view of an embodiment of the drug container, drug channel, and needle assembly of the drug delivery device.

FIG. 12A is a perspective view and FIG. 12B is a cross-sectional view of an embodiment of the drug container, drug channel, and needle assembly of the drug delivery device.

FIG. 13A is a perspective view and FIG. 13B is a cross-sectional view of an embodiment of the drug container, drug channel, and needle assembly of the drug delivery device.

While the invention is amenable to various modifications and alternative forms, specific embodiments have been shown by way of example in the drawings and are described in detail below. The intention, however, is not to limit the invention to the particular embodiments described. On the contrary, the invention is intended to cover all modifications, equivalents, and alternatives falling within the scope of the invention as defined by the appended claims.

### DETAILED DESCRIPTION

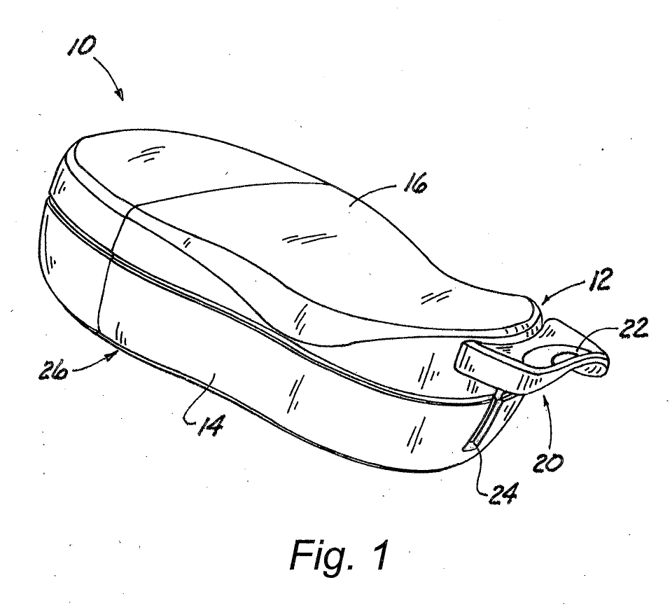

FIGS. 1-13 show various embodiments of the drug delivery device. In all embodiments, the drug container as a whole, not just the drug reservoir, is dynamic. The drug reservoir slides or tips. The drug channel bends, twists, or flexes to accommodate the dynamic movement of the container with the least mechanical resistance, thereby reducing the required activation force when actuating the device. Because only the needle assembly portion of the drug container is constrained, the drug container as a whole is left unconstrained and therefore stress throughout the drug container is continuously redistributed as the needle assembly is actuated from the engaged and unengaged configurations. The distributed stress, during both storage and activation, reduces the chance of material damage, such as fracture, breakage, unfavorable creep, bending, peeling, cracking, fragmenting or the like, that might cause product failure.

FIG. 1 is a perspective view of one embodiment of the drug delivery device 10 in an unengaged configuration. In the unengaged configuration, the needle or other mechanism configured to deliver the drug to tissue, is positioned within or retracted into the drug delivery device 10. The needle and other components of the drug delivery device 10 will be described in more detail herein. The drug delivery device 10 includes a housing 12 that is comprised of a base 14 and a cover 16 coupled together to define an enclosure. When assembled, the base 14 and cover 16 define a top, a bottom, and one or more sidewalls extending between the top and bottom. The drug delivery device 10 further includes a needle actuation mechanism 20 that extends from an end of the housing 12. In some embodiments, the housing 12 and needle actuation mechanism 20 are each comprised of a polymeric material. In other embodiments, the housing 12 and/or needle actuation mechanism 20 are comprised of metal or other suitable materials.

The base 14 includes a bottom surface 26 that is configured to couple with a portion of a body of a user or patient to which the drug is to be delivered. In some embodiments, the bottom surface 26 is substantially planar. In some embodiments, the bottom surface 26 includes an adhesive material that secures the drug delivery device 10 to the target area of the patient. The adhesive material may be covered with a removable adhesive liner that is removed by the user to expose the adhesive when ready to secure to the target area.

The needle actuation mechanism 20 is manually movable from the unengaged configuration shown to an engaged configuration (described in more detail herein) by pressing a handle 22 of the needle actuation mechanism 20 toward the bottom surface of the base 14 (downwardly in FIG. 1). As used herein, the term “manually” means done, performed, or operated by hand or human effort. In some embodiments, the handle 22 is configured to receive a digit (e.g., thumb) of a hand for ease of use. The needle actuation mechanism 20 slides with respect to the housing 12 in a slot 24 defined in the end of the housing 12. In some embodiments, the needle actuation mechanism 20 is lockable in either of the unengaged and engaged configurations, and freely slidable between the two configurations. For example, the slot 24 may include a lip or other feature for retaining the needle actuation mechanism 20 in the unengaged configuration or engaged configuration. The lip or other feature provides sharps protection such that the needle actuation mechanism 20 is disengaged from the lip or other feature prior to sliding the needle actuation mechanism 20 to the engaged position. The shape of slot 24 requires the trigger arm or handle 22 to flex outward given that the resting state of the plastic handle 22 is in the notch. As the handle 22 is moved from the unengaged to the engaged configuration, the handle 22 will fit into position in the slot 24, In some embodiments, this will create an audible snap or other noise so as to provide auditory feedback to the user that device activation has occurred. Further, this slot 24 functionality allows the actuation mechanism 20 to be self-locking, an important aspect of sharps protection.

Additionally, the force required to activate the handle 22 is reduced by the unconstrained bulk of the drug container. The drug reservoir 30 and drug channel 32 are continuously dynamic at different material regions during different travel distances of actuation. This causes their collective resistance to movement to be minimized and in turn, minimizes the force required to activate the drug delivery device. In some embodiments, the force required for needle actuation is less than 10 pound/feet (lbf.), in other embodiments the force required is less than 5 lbf. The reduced activation force maximizes the usability of the device for the general population who might be able to apply only a limited amount of force in order to activate the device.

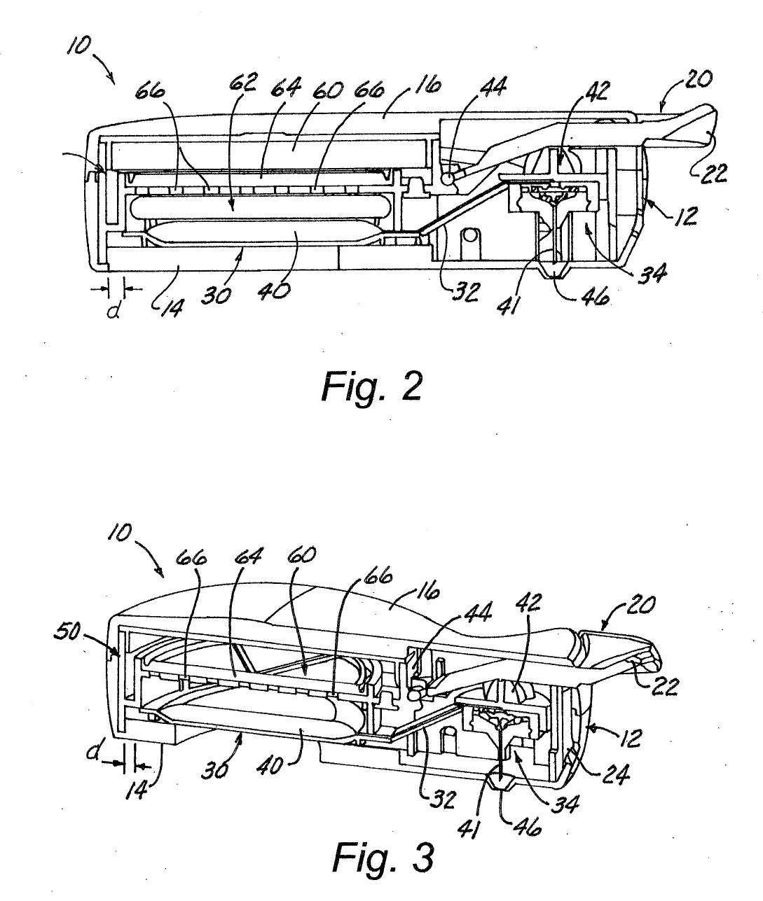

FIG. 2 is a cross-sectional view and FIG. 3 is a perspective cross-sectional view of one embodiment of the drug delivery device 10 in the unengaged configuration. The cross-sectional view shown in FIGS. 2 and 3 is from an imaginary plane extending substantially along the center of the drug delivery device 10. The components within the enclosure of the housing 12 include a drug container 30, a drug channel 32, and a needle assembly 34. The drug container 30 is fluidly connected to the needle assembly 34 via the drug channel 32. In some embodiments, the drug container 30, drug channel 32, and needle assembly 34 are formed as a unitary component (e.g., by thermal bonding and/or snap assembly). Because the drug container 30 is dynamic, the overall length of the drug channel 32 can be minimized, thus reducing the overall size of the drug delivery device 10. This reduction in size has positive effects on wearability and usability of the device 10.

In some embodiments, the drug container 30 comprises a semi-rigid assembly including a drug reservoir 40 that retains the drug to be delivered therein. The drug reservoir 40 may be sized to retain a specific or preferred dosage of the drug. The drug container 30 is configured to collapse upon application of an external force, thereby forcing the drug in the drug reservoir 40 into the drug channel 32. In some embodiments, the drug container 30 is comprised of only non-metal materials. For example, the drug container 30 may be comprised of a thin polymeric film The drug container 30 may also be comprised of biodegradable materials. In other embodiments, the drug container 30 comprises a thin film aluminum layer to prevent evaporation of the drug in the drug reservoir 40 during storage.

The drug channel 32 comprises a fluid conduit that fluidly connects the drug reservoir 40 to the needle assembly 34. In some embodiments, the drug channel 32 comprises a durable, flexible polymeric material, such as polyethylene. In other embodiments, the drug channel 32 is formed by one or more thin films which may be attached to semi-rigid arms to allow for bending, twisting and flexing. The drug channel 32 also mechanically couples the drug container 30 to the needle assembly 34. In the embodiment shown, the drug channel 32 is angled with respect to the major plane of the drug container 30. The drug channel 32 angles with respect to the drug container 30 when the needle actuation mechanism 20 is in the unengaged configuration illustrated in FIGS. 2 and 3. In some embodiments, the drug channel 32 is capable of movement relative to the needle actuation mechanism 20 while the drug reservoir 40 and/or targeted areas of the drug container 30 remain fixed or substantially fixed. The angling of the drug channel 32 in the unengaged configuration allows the overall length of the drug delivery device 10 to be minimized. As will be described in more detail herein, the drug container 30 is movable with respect to the base 14 of the housing 12, which relieves the stress on the drug channel 32 when the needle actuation mechanism 20 is actuated between the unengaged and engaged configurations.

The needle assembly 34 includes a needle 41 that provides a fluid path from the drug reservoir 40 and drug channel 32 to the tissue to which the drug is delivered. In some embodiments, the needle 41 is a straight needle. Alternatively, the needle 41 may be a curved needle or may be replaced with a cannula assembly. In some embodiments, the needle 41 is the only metal component in the drug delivery assembly 10. In other embodiments, the needle 41 is also comprised of a non-metal material. The needle 41 (or, alternatively, the cannula assembly) can be any known device for injecting flowable materials into a patient.

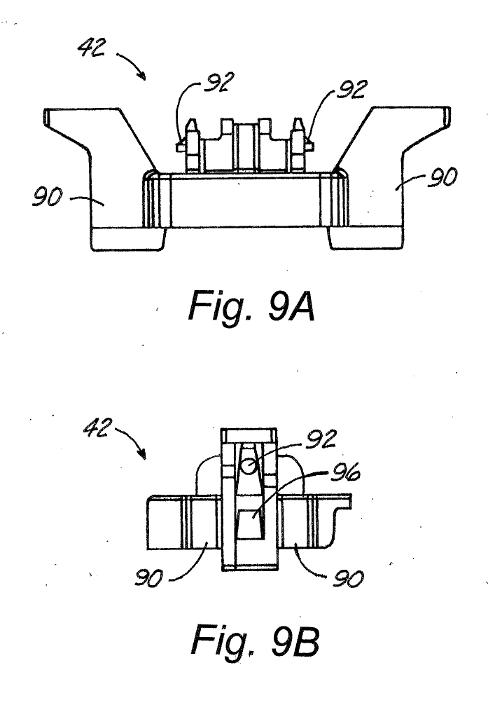

The needle assembly 34 is configured such that the needle 41 translates substantially perpendicular into the tissue when the needle actuation mechanism 20 is actuated from the unengaged configuration to the engaged configuration. In some embodiments, the needle assembly 34 is coupled to the needle actuation mechanism 20 via a needle assembly adapter 42. The adapter 42 is configured to convert the rotational movement of the needle actuation mechanism 20 about pivot 44 to translational motion. The adapter 42 may be coupled to the needle actuation mechanism 20 such that the adapter 42 pivots with respect to the needle actuation mechanism 20 and maintains the orientation of the needle assembly 34 with respect to the needle port 46. An embodiment of the adapter is described below with respect to FIGS. 9A and 9B. In alternative embodiments, the adapter 42 is configured to provide for alternative (e.g., non-perpendicular) needle trajectories and penetration angles.

In some embodiments, the drug delivery device 10 includes a pressure source 50 that is configured to force the drug in the drug container 30 through the drug channel 32 and needle assembly 34. In some embodiments, the drug delivery device 10 is configured to activate the pressure source 50 when the needle actuation mechanism 20 is actuated from the unengaged configuration to the engaged configuration. The pressure source 50 may be any suitable assembly capable of delivering pressure to the drug container 30, including pneumatic, electrical, mechanical (e.g., spring), or chemical assemblies. A spring may be metallic or non-metallic. In some embodiments, the drug container 30 or drug channel 32 further includes a one-way valve to prevent back flow of the drug into the drug container 30.

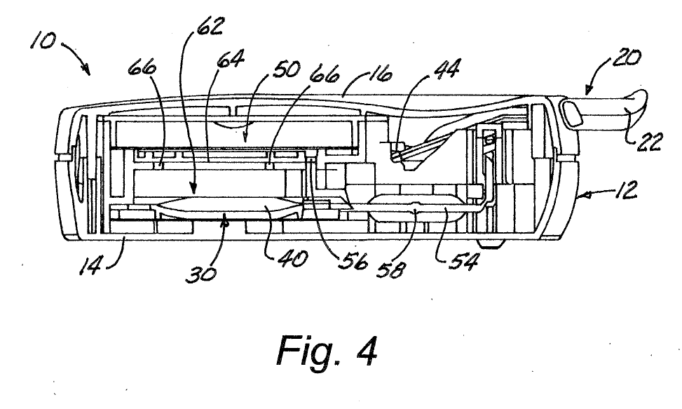

According to another embodiment, the drug delivery device 10 may also include a pressure activation assembly associated with the needle actuation mechanism 20. FIG. 4 is a cross-sectional view of the drug delivery device 10 including a pressure activation assembly 54 configured to activate the pressure source 50. In the embodiment shown, the pressure activation assembly 54 comprises a living hinge. The cross-sectional view shown in FIG. 4 is depicting a portion of the drug delivery device 10 between the cross-sectional plane of FIGS. 2 and 3 and the outer side wall of the drug delivery device 10. In this embodiment, the pressure activation assembly 54 is mechanically coupled to the needle actuation mechanism 20 at one end, and includes a presenting end 56 at the other end. When the needle actuation mechanism 20 is actuated toward the engaged configuration, the pressure activation assembly 54 rotates about pivot 58 and forces the presenting end 56 toward the pressure source 50. The presenting end 56 is configured to activate the pressure source 50 when the needle actuation mechanism 20 is actuated to the engaged configuration.

For example, in the embodiment illustrated in FIGS. 2-4, the pressure source 50 comprises a hydrogel pumping mechanism. The hydrogel pumping mechanism includes a fluid reservoir 60 that retains a fluid for activating the hydrogel. The hydrogel pumping mechanism also includes a hydrogel disc 62 positioned adjacent to the drug container 30. To best illustrate the elements of the hydrogel pumping mechanism, the hydrogel disc 62 is illustrated only in FIG. 2. When the needle actuation mechanism 20 is actuated to the engaged configuration, the presenting end 56 of the pressure activation assembly 54 is urged against a fluid plug 64 of the fluid reservoir 60 to release the fluid plug 64, thereby releasing fluid from the fluid reservoir 60. The released fluid is directed toward the hydrogel disc 62 via apertures 66 in an interface between the fluid reservoir 60 and hydrogel disc 62. In response to being exposed to fluid, the hydrogel disc 62 expands and provides pressure onto the drug container 30. The expansion of the hydrogel disc 62 urges the drug in the drug container 30 through the drug channel 32 and the needle 41 of the needle assembly 34. The drug then exits the needle 41 and is delivered to the individual. In some embodiments, the hydrogel pumping mechanism is configured to expand until substantially all of the drug in the drug container 30 is delivered. In one exemplary implementation, the hydrogel pumping mechanism may be configured similar to the mechanism described in U.S. Patent App. Pub. No. 2010/0030198, entitled “Drug Delivery Platform Utilizing Hydrogel Pumping Mechanism,” which is incorporated herein in its entirety for all purposes.

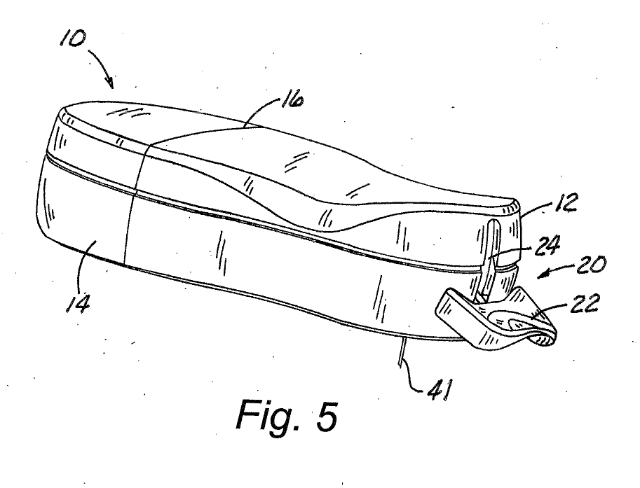

In use, when the individual has positioned and secured the drug delivery device 10 on the desired area of the patient's body for drug delivery, the needle actuation mechanism 20 may be manually actuated from the unengaged configuration to the engaged configuration. In some embodiments, the needle actuation mechanism 20 is manually translated laterally with respect to the slot to disengage the needle actuation mechanism 20 from the unengaged configuration. FIG. 5 is a perspective view of the drug delivery device 10 showing the needle actuation mechanism 20 in the engaged configuration. As is shown, the handle 22 is positioned near the bottom of slot 24. The slot 24 may include a lip or other feature that retains the needle actuation mechanism 20 in the engaged configuration to prevent unintentional withdrawal of the needle 41 during after engagement. In some embodiments, the drug delivery device 10 provides auditory feedback (e.g., a click) and/or tactile feedback to the user to indicate that the needle actuation mechanism 20 is fully in the engaged configuration. The needle 41 extends from the needle port 46 in the base 14 when the needle actuation mechanism 20 is in the engaged configuration. The forces and motion to manually actuate the needle actuation mechanism 20 to the engaged configuration and to activate the pressure source 50 are ergonomically and physically simple

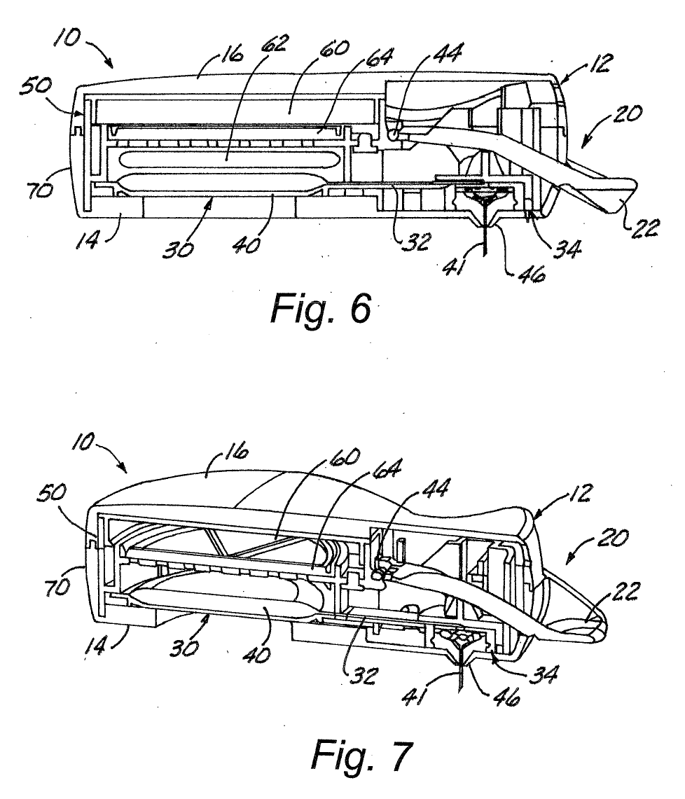

FIG. 6 is a cross-sectional view and FIG. 7 is a perspective cross-sectional view of the drug delivery device 10 in the engaged configuration. In the engaged configuration, the needle assembly 34 is moved toward the base 14. The drug channel 32, in fluid and mechanical communication with the needle assembly 34, moves from an angled configuration with respect to the bottom surface of the base 14 (FIGS. 2 and 3) to an arrangement that is substantially parallel with the bottom surface of the base 14. To accommodate the repositioning of the drug channel 32, according to an embodiment, the drug container 30 is configured to translate along the bottom surface of the base 14 toward a wall 70 of the housing 12 opposite the location of the handle 22. A comparison of the drug delivery device 10 in the unengaged configuration (FIGS. 2 and 3) and the drug delivery device 10 in the engaged configuration (FIGS. 6 and 7) illustrates that the drug container 30 shifts from a distance d from the wall 70 in the unengaged configuration to a near zero distance to the wall 70 in the engaged configuration. The ability of the drug container 30 to slide with respect to the base 14 relieves the stress on the drug channel 32 when the drug delivery device 10 is stored in the unengaged configuration. In addition, the slidability of the drug container 30 allows for a minimization in the length in the drug delivery device 10, since excess length of material is not needed to relieve the stress on the drug channel 32. This reduction in stress on the drug channel 32 reduces the likelihood of material failure of the drug channel 32 during storage. Further, in some embodiments, the flexibility or pliability of the drug channel 32 allows for decreased resistance dynamic movement of the drug container 30.

In some embodiments, the housing 12 includes features that guide the drug container 30 along the base 14. For example, the base 14 may be configured with guides that interface with features formed on the exterior of the drug container 30. The interfacing of these elements may be configured to cause the drug container 30 to move in substantially one dimension (i.e., toward and away from the wall 70).

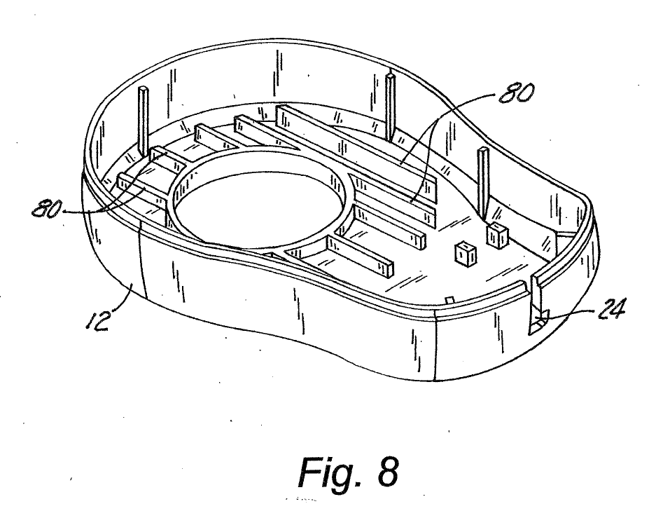

For example, in one exemplary implementation illustrated in FIG. 8, the base 14 includes a plurality of guide rails 80 that extend from the bottom interior surface of the base 14. The guide rails 80 may be configured to interface with protrusions (not shown) on the bottom of the drug container 30. The protrusions on the drug container 30 may be sized and shaped to fit between adjacent guide rails 80 such that the drug container 30 does not move perpendicular to the guide rails 80 when the needle actuation mechanism 20 is actuated between the unengaged and engaged configurations. The guide rails 80 may further include a lip or other feature that prevents the drug container from moving away from the base 12.

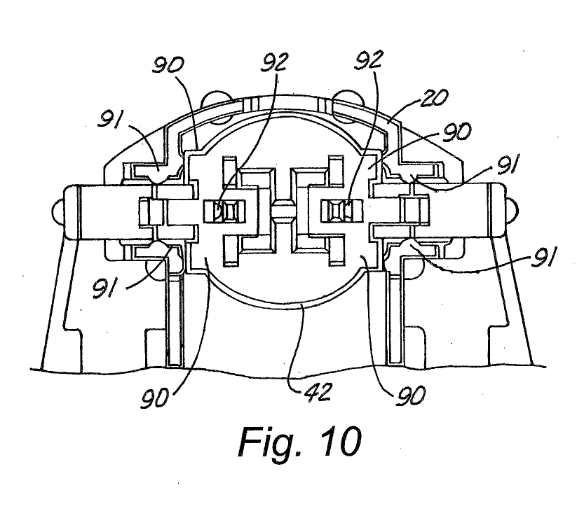

As discussed above, in order to convert the rotational motion of the needle actuation mechanism 20 (as handle 22 is rotated about the pivot 44) to translational motion of the needle 41, the needle assembly 34 is attached to the needle assembly adapter 42, which in turn is coupled to the needle actuation mechanism 20. FIG. 9A is a front view and FIG. 9B is a side view of an embodiment of the needle assembly adapter 42. FIG. 10 is a cross-sectional view illustrating an embodiment of the coupling relationship between the needle assembly adapter 42 and needle actuation mechanism 20. The needle assembly adapter 42 includes a plurality of alignment wings 90 that are configured to interface with corresponding alignment tracks 91 located within the housing 12. The needle assembly adapter 42 also includes pivot protrusions 92 extending the portion that interfaces with the needle actuation mechanism 20. The pivot protrusions 92 couple with the pivot apertures on the needle actuation mechanism 20 to allow the needle assembly adapter 42 to rotate or pivot with respect to the needle actuation mechanism 20. The pivoting of the needle assembly adapter 42 with respect to the needle actuation mechanism 20 corresponds to the rotational motion of the handle 22, thereby converting the rotational motion of the handle 22 to translational motion of the needle assembly 34 (and needle 41).

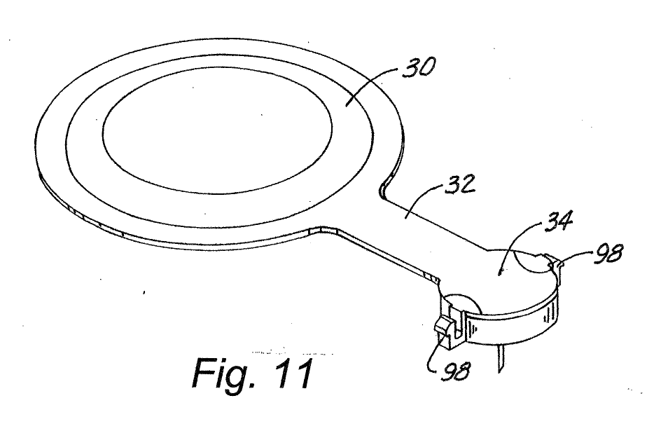

The needle assembly adapter 42 can further include wing snap receiving apertures 96 configured to receive features on the needle assembly 34. FIG. 11 illustrates the drug delivery elements of the drug delivery device 10, including the drug container 30, the drug channel 32, and the needle assembly 34. The needle assembly 34 includes wing snap features 98 that are configured to mate or interface with the wing snap receiving apertures 96 to couple the needle assembly 34 to the needle assembly adapter 42. The arrangement of the needle assembly adapter 42 and the needle assembly 34 allows the needle 41 to travel in a perpendicular direction.

When the drug has been delivered from the drug container 30 to the user, the user may then actuate the needle actuation mechanism 20 from the engaged configuration to the unengaged configuration. The user may then disengage the drug delivery device 10 and, in the case of a single use device, dispose of the drug delivery device 10. In some embodiments, most or all of the components of the drug delivery device 10 are comprised of a biodegradable material.

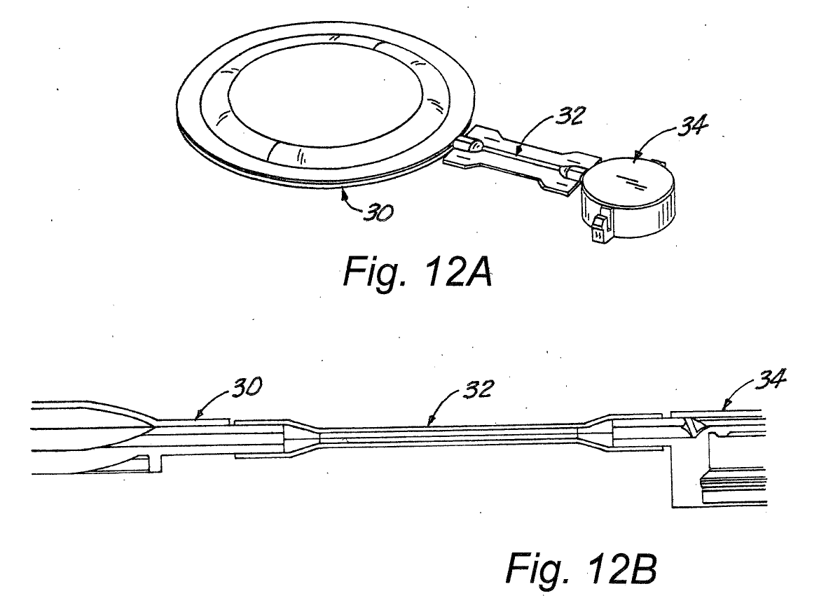

As discussed above, according to another embodiment, the drug delivery device 10 may also possesses a drug channel 32 formed by paired pieces of thin film or other pliable or flexible material. FIGS. 12A and 12B illustrate an embodiment of the drug delivery device 10, including the drug container 30, where a thin film is bonded to another thin film to form the drug channel 32. This allows the drug channel 32 to bend, flex and twist in response to the relative movement of the drug container 30 and reduce the overall resistance to motion, thereby reducing the amount of force required to actuation the needle actuation mechanism.

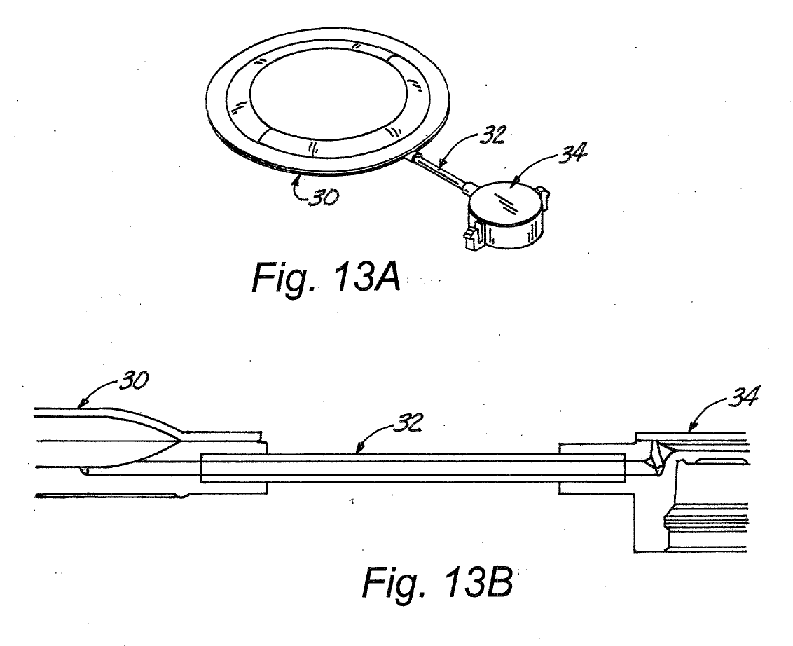

FIGS. 13A and 13B illustrate an embodiment wherein tubing is bonded to the drug reservoir 30 and needle assembly 34. In this embodiment, the drug channel 32 is free to bend, flex, or twist in a non-linear fashion. In these embodiments, the tubing is bonded via adhesive, however, other bonding techniques may be applied, as one of skill in the art would easily recognize.

Various modifications and additions can be made to the exemplary embodiments discussed without departing from the scope of the present invention. For example, while the embodiments described above refer to particular features, the scope of this invention also includes embodiments having different combinations of features and embodiments that do not include all of the above described features.

I claim:

1. A drug delivery device for delivering a drug to an individual, the drug delivery device comprising:
a housing including a base and a cover that define an enclosure;
a drug container disposed within the enclosure, the drug container comprising a drug reservoir configured to retain the drug therein, the drug container including external features configured to allow for dynamic movement relative to the housing;
a needle assembly including a needle;
a drug channel fluidly connected between the drug container and needle assembly; and
a needle actuation mechanism coupled to the needle assembly and actuatable between an unengaged configuration and an engaged configuration, wherein the needle is extended from the housing in the engaged configuration and retracted in the housing in the unengaged configuration, and wherein the drug container moves dynamically relative to the housing to relieve stress on the drug container when the needle actuation mechanism is actuated between the engaged and unengaged configurations.

2. The drug delivery device of claim 1, wherein a major axis of the drug channel is substantially parallel to a major surface of the drug container when the needle actuation mechanism is in the engaged configuration, and wherein the major axis of the drug channel is non-zero angled with respect to the major surface of the drug container when the needle actuation mechanism is in the unengaged configuration.

3. The drug delivery device of claim 1, and further comprising:
a pressure source engagable with the drug container for urging the drug from the reservoir through the needle assembly via the drug channel

4. The drug delivery device of claim 3, and further comprising:
a pressure activation assembly coupled to the needle actuation mechanism, wherein actuation of the needle actuation mechanism to the engaged configuration causes a presenting end of the pressure activation assembly to activate the pressure source.

5. The drug delivery device of claim 4, wherein the pressure source comprises a hydrogel that expands in response to exposure to a fluid.

6. The drug delivery device of claim 5, wherein actuation of the needle actuation mechanism to the engaged configuration causes the presenting end to release fluid that activates the hydrogel.

7. The drug delivery device of claim 1, wherein the needle actuation mechanism rotates about an axis when actuated between the unengaged and engaged configurations.

8. The drug delivery device of claim 7, wherein the needle actuation mechanism comprises an adapter that couples to the needle assembly and is configured to convert rotational motion of the needle actuation mechanism to translational motion of the needle assembly.

9. The drug delivery device of claim 1, wherein the drug channel comprises a flexible polymeric material.

10. The drug delivery device of claim 1, wherein the needle is the only metallic component of the drug delivery device.

11. The drug delivery device of claim 1, wherein the needle actuation mechanism comprises an ergonomic lever that is lockable in the engaged and unengaged configurations and slidable between the engaged and unengaged configurations.

12. The drug delivery device of claim 12, wherein the ergonomic lever is self-locking.

13. A drug delivery device for delivering a drug to an individual, the drug delivery device comprising:
a housing defining an enclosure having a top surface, a bottom surface, and one or more side walls extending between the top and bottom surfaces;
a drug container disposed within the enclosure, the drug container comprising a drug reservoir for retaining the drug therein, the drug container configured to cooperate with the housing to allow for dynamic movement of the drug reservoir relative to the housing;
a needle assembly including a needle;
a drug channel fluidly connected between the drug container and needle assembly; and
a needle actuation mechanism coupled to the needle assembly and actuatable between an engaged configuration and an unengaged configuration, wherein the needle is extended from the housing in the engaged configuration and the needle is retracted in the housing in the unengaged configuration, and wherein the drug container is substantially unconstrained within the drug delivery device to relieve stress on the drug container when the needle actuation mechanism is actuated between the engaged and unengaged configurations.

14. The drug delivery device of claim 13, wherein a major axis of the drug channel is substantially parallel to a major surface of the drug container when the needle actuation mechanism is in the engaged configuration, and wherein the major axis of the drug channel is non-zero angled with respect to the major surface of the drug container when the needle actuation mechanism is in the unengaged configuration.

15. The drug delivery device of claim 13, and further comprising:
a pressure source engagable with the drug container for urging the drug from the reservoir through the needle assembly via the drug channel.

16. The drug delivery device of claim 15, and further comprising:
a pressure activation assembly coupled to the needle actuation mechanism, wherein actuation of the needle actuation mechanism to the engaged configuration causes a presenting end of the pressure activation assembly to activate the pressure source.

17. The drug delivery device of claim 16, wherein the pressure source comprises a hydrogel that expands in response to exposure to a fluid.

18. The drug delivery device of claim 17, wherein actuation of the needle actuation mechanism to the engaged configuration causes the presenting end to release fluid that activates the hydrogel.

19. The drug delivery device of claim 13, wherein the needle actuation mechanism rotates about an axis when actuated between the unengaged and engaged configurations.

20. The drug delivery device of claim 19, wherein the needle actuation mechanism comprises an adapter that couples to the needle assembly and is configured to convert rotational motion of the needle actuation mechanism to translational motion of the needle assembly.

21. The drug delivery device of claim 13, wherein the needle actuation mechanism comprises an ergonomic lever that is lockable in the engaged and unengaged configurations and freely slidable between the engaged and unengaged configurations.

22. The drug delivery device of claim 13, wherein the drug channel comprises a flexible polymeric material.

23. The drug delivery device of claim 13, wherein the needle is the only metallic component of the drug delivery device.

24. A drug delivery device for delivering a drug to an individual, the drug delivery device comprising:
a housing defining an enclosure;
a drug container disposed within the enclosure capable of movement within the disclosure, the drug container comprising a drug reservoir for retaining the drug therein;
a needle assembly including a needle;
a drug channel fluidly connected between the drug container and needle assembly;
a pressure source engagable with the drug container for urging the drug from the reservoir through the needle assembly via the drug channel;
a needle actuation mechanism coupled to the needle assembly and actuatable between an engaged configuration and an unengaged configuration, wherein the needle is extended from the housing in the engaged configuration and retracted in the housing in the unengaged configuration, and wherein the needle actuation mechanism is lockable in the engaged and unengaged configurations and slidable between the engaged and unengaged configurations; and
a pressure activation assembly coupled to the needle actuation mechanism, wherein actuation of the needle actuation mechanism to the engaged configuration causes a presenting end of the pressure activation assembly to activate the pressure source.

24. The drug delivery device of claim 24, wherein the activation button's natural state is in the lockable engaged or unengaged configuration, and the ergonomic lever is self-locking

25. The drug delivery device of claim 25, wherein the self-locking aspect of the ergonomic lever is overcome with minimal activation force and the movement from the engaged and unengaged positions is reliably guided by the slot geometry.

26. The drug delivery device of claim 24, wherein the pressure source comprises a hydrogel that expands in response to exposure to a fluid.

27. The drug delivery device of claim 26, wherein actuation of the needle actuation mechanism to the engaged configuration causes the presenting end to release fluid that activates the hydrogel.
